(19) United States
(12) Patent Application Publication
(10) Pub. No.: US 2015/0202233 A1
(43) Pub. Date: Jul. 23, 2015

(54) METHOD TO GENERATE TISSUE-ENGINEERED CARTILAGE IN ULTRASONIC BIOREACTORS

(71) Applicant: NUtech Ventures, Lincoln, NE (US)

(72) Inventors: Anuradha Subramanian, Lincoln, NE (US); Hendrik Viljoen, Lincoln, NE (US); Tobi Louw, Fishhoek (ZA)

(21) Appl. No.: 14/603,013

(22) Filed: Jan. 22, 2015

Related U.S. Application Data
(60) Provisional application No. 61/930,061, filed on Jan. 22, 2014.

(51) Int. Cl. A61K 35/32; C12N 13/00

(52) U.S. Cl. CPC A61K 35/32; C12N 13/00; C12N 2506/03

(57) ABSTRACT

The present disclosure describes methods of using ultrasound to engineer cartilage, as well as a bioreactor that includes at least one ultrasound transducer.

### CROSS REFERENCE TO RELATED APPLICATIONS

This application claims the benefit of priority under 35 U.S.C. 119(e) to U.S. Application No. 61/930,061 filed Jan. 22, 2014.

### TECHNICAL FIELD

This disclosure generally relates to engineering cartilage.

### BACKGROUND

Ultrasound uses high-frequency sound waves to image objects and measure distances.

### SUMMARY

In one aspect, a method of engineering cartilage is provided. Such a method typically includes exposing stem cells to continuous low-intensity ultrasound to produce chondrocytes.

In another aspect, a method of engineering cartilage is provided. Such a method typically includes exposing stem cells to continuous low-intensity ultrasound to produce chondrocytes; and implanting the chondrocytes into a patient.

In one embodiment, the stem cells are exposed to the continuous low-intensity ultrasound in culture. In one embodiment, the stem cells are seeded on a scaffold structure. In one embodiment, the scaffold structure is a focal defect-sized scaffold. In one embodiment, the scaffold structure comprises poly(lactic-co-glycolic acid) (PLGA) copolymer. In one embodiment, the culture includes growth factors

In one embodiment, the primary resonant frequency of the continuous low-intensity ultrasound includes from about 4.5 MHz to about 6.0 MHz. For example, a representative primary resonant frequency of the continuous low-intensity ultrasound is about 5.2 MHz. In some embodiment, the secondary resonant frequency of the continuous low-intensity ultrasound includes about 8.0 MHz to about 10.5 MHz. For example, a representative secondary resonant frequency of the continuous low-intensity ultrasound includes about 9.5 MHz.

In some embodiment, the continuous low-intensity ultrasound includes a pressure of about 10 kPa to about 120 kPa. In some embodiments, the continuous low-intensity ultrasound includes a duration of exposure of about 1 to about 20 minutes. Representative stem cells include, without limitation, hMSC, fibroblast, osteoblast, iPSCs.

In yet another aspect, a bioreactor for engineering cartilage is provided. Typically, the bioreactor includes at least one ultrasonic transducer configured to provide continuous low-intensity ultrasound to stem cells on a scaffold structure and in culture.

In one embodiment, the scaffold structure is placed above the at least one ultrasonic transducer. In one embodiment, the dimensions of the scaffold structure are approximately the same as that of the at least one ultrasonic transducer. In some embodiments, a culture plate that includes the cells is in fluid communication with the at least one ultrasonic transducer.

In some embodiments, the bioreactor comprises at least two ultrasonic transducers configured to provide continuous low-intensity ultrasound to the cells during culture. In some embodiments, each of the at least two ultrasonic transducers is configured to deliver different frequencies and/or different pressures of continuous low-intensity ultrasound to the cells during culture.

In some embodiments, such a bioreactor can include a positioning stage upon which the culture plate is seated, wherein the positioning stage allows for changing the distance between the at least one ultrasound transducer and the cells comprised within the culture plate. In some embodiments, the bioreactor also can include a microprocessor. In some embodiments, the stem cells are selected from the group consisting of hMSC, fibroblast, osteoblast, and iPSC.

Unless otherwise defined, all technical and scientific terms used herein have the same meaning as commonly understood by one of ordinary skill in the art to which the methods and compositions of matter belong. Although methods and materials similar or equivalent to those described herein can be used in the practice or testing of the methods and compositions of matter, suitable methods and materials are described below. In addition, the materials, methods, and examples are illustrative only and not intended to be limiting. All publications, patent applications, patents, and other references mentioned herein are incorporated by reference in their entirety.

### DESCRIPTION OF DRAWINGS

The figures and the corresponding descriptions can be found in each of the Appendices.

### DETAILED DESCRIPTION

This disclosure describes the ability of ultrasound stimulation to impact the proliferative and biosynthetic activity of cells in culture. The methods described herein are directed toward exposing cells in culture to continuous low-intensity ultrasound.

The particular ultrasound conditions will be dependent upon the particular cells, the particular culture conditions and scaffold structure used, and the desired outcome of the ultrasound exposure. As described herein, under typical culture conditions (e.g., mammalian chondrocytes with a scaffold structure (e.g., a polymer, e.g., PLGA, PLA, PGA)), low-intensity-ultrasound conditions include a primary frequency of from about 4.5 MHz to about 6.0 MHz (e.g., about 5.0 MHz to about 6 MHz, about 5.5 MHz to about 6.0 MHz, about 4.5 MHz to about 5 MHz, or about 5.2 MHz) and a pressure of about 10 kPa to about 120 kPa (e.g., about 10 kPa to about 100 kPa, about 14 kPa to about 80 kPa, about 15 kPa to about 60 kPa, about 25 kPa to about 50 kPa, about 30 kPa to about 60 kPa, about 35 kPa to about 55 kPa, about 40 kPa to about 50 kPa, about 45 kPa to about 55 kPa, about 15 kPa to about 25 kPa, about 15 kPa to about 30 kPa, about 20 kPa to about 30 kPa, about 25 kPa to about 40 kPa, about 30 kPa to about 50 kPa, about 35 kPa to about 50 kPa, about 40 kPa to about 60 kPa, about 45 kPa to about 60 kPa, or about 50 kPa to about 60 kPa). In some instances, a secondary frequency of from about 8.0 MHz to about 10.5 MHz (e.g., about 8.5 MHz to about 10 MHz, about 9 MHz to about 10.5 MHz, about 9 MHz to about 10 MHz, or about 9.5 MHz) can be used.

As used herein, an intermittent low-intensity-diffuse ultrasound signal can be delivered for a duration or length of time of from about 1.0 min to about 20 mins (e.g., about 1.0 min to about 15 mins, about 5 min to about 20 mins, about 1 min to about 15 mins, about 1 min to about 10 mins, about 2 mins to about 5 mins, about 2 mins to about 8 mins, about 3 mins to about 5 mins, about 3 mins to about 7 mins, about 4 mins to about 9 mins, about 4 mins to about 7 mins, about 5 mins to about 10 mins, about 5 mins to about 8 mins, about 6 mins to about 8 mins, about 6 mins to about 10 mins, about 6 mins to about 9 mins, about 7 mins to about 10 mins, about 7 mins to about 9 mins, or about 8 mins to about 10 mins). In addition, a continuous low-intensity ultrasound signal can be delivered at an interval of from about 2 to 8 times per day (e.g., once every 3 hours, once every 4 hours, once every 5 hours, once every 6 hours, once every 10 hours, once every 12 hours, once every 18 hours, once every 24 hours) up to about 20 or more times per day (e.g., once every hour, once every 2 hours, once every 6 hours, once every 8 hours, once every 10 hours).

The continuous low-intensity ultrasound described herein is not limited to any particular types of stem or progenitor cells. Simply by way of example, suitable cells include, without limitation, osteoblasts, fibroblasts, and stem cells (e.g., mesenchymal stem cells (MSCs), embryonic stem cells (ESCs), induced pluripotent stem cells (iPSCs)). Simply by way of example, bone and cartilage often is irreversibly destroyed following traumatic injury or due to chronic illnesses such as arthritis. Since neither bone nor cartilage exhibits significant self-repair, a promising alternative therapy is the transplantation of tissue-engineered bone or cartilage. The continuous low-intensity ultrasound described herein is particularly useful for engineering cartilage.

The continuous low-intensity ultrasound described herein can be incorporated into a culture system (e.g., a bioreactor) such that cells or tissues can be exposed to ultrasound in culture. Bioreactors are well known, and generally refer to a device that supports and maintains the viability of cells or tissues in culture and, in some instances, promotes the biological growth and/or development of the cells or tissues. In some embodiments, a tissue culture plate can be positioned directly above a cavity containing at least one ultrasound transducer. It would be understood that any number of ultrasound transducers can be used in an ultrasonic bioreactor as described herein, provided that the transducers deliver the appropriate strength and pressure of signal to the cells or tissue. In some embodiments, enclosing the transducers within a box may be desired, as there may be configurations in which continuous low-intensity ultrasound is delivered to cells more effectively in the absence of a cavity.

An ultrasonic bioreactor also can include a positioning stage. In some embodiments, the positioning stage is below the one or more ultrasound transducers, such that the box containing the ultrasound transducers can be moved in any of the x-, y- or z-axes relative to the tissue culture plate. In some embodiments, however, the positioning stage also can be located above the ultrasound transducers but below the tissue culture plate. This configuration would allow for movement of the tissue culture plate in the x-, y- or z-axes relative to the ultrasound transducers. In whatever configuration, a positioning stage provides one mechanism by which the distance between the cells and the ultrasound transducer can be changed, which ultimately provides a mechanism by which the frequency and/or pressure applied to the cells can be changed. It would be appreciated that the actual position of the ultrasound transducers relative to the tissue culture plates is less relevant than the actual ultrasound signal strength and pressure applied to the cells.

It would be understood that a bioreactor also can include a splitter for controlling the signal sent to each transducer. A bioreactor as described herein also can include a microprocessor to control the components of the bioreactor, although it would be appreciated that a microprocessor can be provided without the additional components provided by a computer (e.g., screen, keyboard, etc.).

Those skilled in the art would appreciate that at least one ultrasonic transducer can be incorporated into an existing or conventional (e.g., commercially available) bioreactor. Alternatively, a bioreactor can be specifically designed to include, in addition to the other components typically found in a bioreactor, at least one ultrasonic transducer. Those skilled in the art also would appreciate that any configuration of the one or more ultrasound transducers with respect to the cells or tissues in a bioreactor is suitable provided that the culture (i.e., the cells) can be exposed to the appropriate strength and/or pressure of signal for the appropriate duration. As described herein, there are certain advantages when the ultrasonic transducer is in fluid communication with the tissue culture plate that contains the cells or with a structure that holds or supports the tissue culture plate (e.g., a positioning stage). In some embodiments, one or more ultrasonic transducer can be positioned within a fluid-filled structure or cavity that is in contact with or in communication with the tissue culture plate or a structure holding or supporting the tissue culture plate.

It would be understood by those in the art that more than one transducer can be used (e.g., two, three, four, five, six, or more transducers) to expose cells to ultrasound. More than one transducer can be used to deliver an ultrasound signal to a larger surface area than could be delivered by a single transducer. Additionally or alternatively, more than one transducer can be used to deliver different continuous low-density ultrasound signals to the cells during culture (e.g., a gradient of signals). For example, different transducers can deliver different frequencies and/or different pressures of ultrasound signal to the culture and/or different transducers can deliver ultrasound signals (e.g., the same or different) for different durations of time and/or different intervals between signals.

In accordance with the present invention, there may be employed conventional molecular biology, microbiology, biochemical, and recombinant DNA techniques within the skill of the art. Such techniques are explained fully in the literature. The invention will be further described in the following examples, which do not limit the scope of the methods and compositions of matter described in the claims.

### EXAMPLES

### Example 1

### Combined Transfer Matrix/Angular Spectrum Approach Applied to Layered Media for Computationally Efficient Acoustic Field Simulation

See Appendix A

### Example 2

### Enhanced Depth-Dependent Cellular Colonization of Articular Chondrocytes and Expression of Chondrocytic Markers under Ultrasound Stimulation in an Ultrasound-Assisted Bioreactor

See Appendix B

### Example 3

### Design and Control of Ultrasonic Bioreactors to Ensure Experimental Repeatability

See Appendix C

### Example 4

### Mechanotransduction of Ultrasound is Frequency Dependent Below the Cavitation Threshold

See Appendix D

### Example 5

### Ultrasonic Stimulation of Chondrocytes: Harmonic Analysis Elucidates Frequency Dependent Bioeffects

See Appendix E

It is to be understood that, while the methods and compositions of matter have been described herein in conjunction with a number of different aspects, the foregoing description of the various aspects is intended to illustrate and not limit the scope of the methods and compositions of matter. Other aspects, advantages, and modifications are within the scope of the following claims.

Disclosed are methods and compositions that can be used for, can be used in conjunction with, can be used in preparation for, or are products of the disclosed methods and compositions. These and other materials are disclosed herein, and it is understood that combinations, subsets, interactions, groups, etc. of these methods and compositions are disclosed. That is, while specific reference to each various individual and collective combinations and permutations of these compositions and methods may not be explicitly disclosed, each is specifically contemplated and described herein. For example, if a particular composition of matter or a particular method is disclosed and discussed and a number of compositions or methods are discussed, each and every combination and permutation of the compositions and the methods are specifically contemplated unless specifically indicated to the contrary. Likewise, any subset or combination of these is also specifically contemplated and disclosed.

What is claimed is:

1. A method of engineering cartilage, comprising:
exposing stem cells to continuous low-intensity ultrasound to produce chondrocytes.

2. A method of engineering cartilage, comprising:
exposing stem cells to continuous low-intensity ultrasound to produce chondrocytes; and
implanting the chondrocytes into a patient.

3. The method of claim 1 or 2, wherein the stem cells are exposed to the continuous low-intensity ultrasound in culture.

4. The method of claim 1 or 2, wherein the stem cells are seeded on a scaffold structure.

5. The method of claim 4, wherein the scaffold structure is a focal defect-sized scaffold.

6. The method of claim 4, wherein the scaffold structure comprises poly(lactic-co-glycolic acid) (PLGA) copolymer.

7. The method of claim 3, wherein the culture includes growth factors

8. The method of claim 1 or 2, wherein the primary resonant frequency of the continuous low-intensity ultrasound comprises from about 4.5 MHz to about 6.0 MHz.

9. The method of claim 1 or 2, wherein the primary resonant frequency of the continuous low-intensity ultrasound comprises about 5.2 MHz.

10. The method of claim 1 or 2, wherein the secondary resonant frequency of the continuous low-intensity ultrasound comprises about 8.0 MHz to about 10.5 MHz.

11. The method of claim 1 or 2, wherein the secondary resonant frequency of the continuous low-intensity ultrasound comprises about 9.5 MHz.

12. The method of claim 1 or 2, wherein the continuous low-intensity ultrasound comprises a pressure of about 10 kPa to about 120 kPa.

13. The method of claim 1 or 2, wherein the continuous low-intensity ultrasound comprises a duration of exposure of about 1 to about 20 minutes.

14. The method of claim 1 or 2, wherein the stem cells are selected from the group consisting of hMSC, fibroblast, osteoblast, iPSCs.

15. A bioreactor for engineering cartilage, wherein the bioreactor comprises at least one ultrasonic transducer configured to provide continuous low-intensity ultrasound to stem cells on a scaffold structure and in culture.

16. The bioreactor of claim 15, wherein the scaffold structure is placed above the at least one ultrasonic transducer.

17. The bioreactor of claim 15, wherein the dimensions of the scaffold structure are approximately the same as that of the at least one ultrasonic transducer.

18. The bioreactor of claim 15, wherein a culture plate comprising the cells is in fluid communication with the at least one ultrasonic transducer.

19. The bioreactor of claim 15, wherein the bioreactor comprises at least two ultrasonic transducers configured to provide continuous low-intensity ultrasound to the cells or tissues during culture.

20. The bioreactor of claim 19, wherein each of the at least two ultrasonic transducers is configured to deliver different frequencies and/or different pressures of continuous low-intensity ultrasound to the cells or tissue during culture.

21. The bioreactor of claim 15, further comprising a positioning stage upon which the culture plate is seated, wherein the positioning stage allows for changing the distance between the at least one ultrasound transducer and the cells comprised within the culture plate.

22. The bioreactor of claim 15, further comprising a microprocessor.

23. The bioreactor of claim 15, wherein the stem cells are selected from the group consisting of hMSC, fibroblast, osteoblast, and iPSC.
